(19) United States
(12) Patent Application Publication
(10) Pub. No.: US 2010/0316727 A1
(43) Pub. Date: Dec. 16, 2010

(54) TREATMENT OF INFLAMMATORY DISORDERS WITH OZONE

(76) Inventors: Joseph S. Latino, Brooklyn, NY (US); Steven A. Keyser, Salt Lake City, UT (US)

Correspondence Address: MORRISS OBRYANT COMPAGNI, P.C., 734 EAST 200 SOUTH, SALT LAKE CITY, UT, 84102, US

(21) Appl. No.: 12/819,886

(22) Filed: Jun. 21, 2010

Related U.S. Application Data
(63) Continuation-in-part of application No. 12/813,371, filed on Jun. 10, 2010.
Application No. 12/813,371 is a division of application No. 10/963,477, filed on Oct. 11, 2004, now Pat. No. 7,736,494.
Application No. 10/963,477 is a continuation-in-part of application No. 10/910,485, filed on Aug. 2, 2004, now abandoned.
(60) Provisional application No. 61/269,090, filed on Jun. 19, 2009.
(60) Provisional application No. 60/553,774, filed on Mar. 17, 2004.
(60) Provisional application No. 60/491,997, filed on Jul. 31, 2003.

(51) Int. Cl. A61K 33/40; A61K 35/14; A61K 35/16; A61P 29/00; A61P 9/08; A61P 37/02; A61P 3/10; A61P 1/00; A61P 17/06; A61P 9/10; A61P 25/28; A61P 25/00; A61P 25/24; A61P 19/02; A61P 11/00; A61P 25/16

(52) U.S. Cl. 424/532; 424/613; 424/529; 424/530

(57) ABSTRACT

Methods for therapeutic treatment of inflammatory conditions in a mammalian patient provide clinical benefits including reduction of inflammation, vasorelaxation, reduction in edema and increased blood flow, the methods generally comprising extracorporeal treatment of blood, blood fractionate, or other biological fluid to expose such fluids to a precise, measured amount of ozone to produce a blood or biological fluid having a quantified absorbed dose of ozone, and reinfusing the treated biological fluid to the patient to provide therapeutic effects beneficial in the treatment of inflammatory disorders and related symptoms or conditions.

### CROSS REFERENCE TO RELATED APPLICATIONS

This is a non-provisional application which claims priority to provisional Ser. No. 61/269,090, filed Jun. 19, 2009, and this application also claims priority to co-pending Ser. No. 12/813,371, filed Jun. 10, 2010, which is a divisional application of Ser. No. 10/963,477, filed Oct. 11, 2004, which is a continuation-in-part of Ser. No. 10/910,485, filed Aug. 2, 2004, which claims priority to both provisional application Ser. No. 60/553,774, filed Mar. 17, 2004, and provisional application Ser. No. 60/491,997, filed Jul. 31, 2003. The contents of all foregoing applications are incorporated herein, in their entirety, by reference.

### BACKGROUND OF THE INVENTION

1. Field of the Invention

This invention relates to therapeutic treatments for inflammatory disorders and related medical conditions in mammals, and specifically relates to therapeutic treatment of inflammatory disorders or conditions in a mammalian patient using quantifiable absorbed doses of ozone delivered to a biological fluid by an ozone delivery system.

2. Statement of the Related Art

The references discussed herein are provided solely for the purpose of describing the field relating to the invention. Nothing herein is to be construed as an admission that the inventors are not entitled to antedate a disclosure by virtue of prior invention. Furthermore, citation of any document herein is not an admission that the document is prior art, or considered material to patentability of any claim herein, and any statement regarding the content or date of any document is based on the information available to the applicant at the time of filing and does not constitute an affirmation or admission that the statement is correct.

Apoptosis specifically refers to an energy-dependent, asynchronous, genetically controlled process by which unnecessary or damaged single cells self-destruct when apoptosis genes are activated (Martin, S J 1993; Earnshaw, W C 1995). There are three distinct phases of apoptosis. Initially, the cell shrinks and detaches from neighboring cells. The nucleus is broken down with changes in DNA including strand breakage (karyorhexis) and condensation of nuclear chromatin (pyknosis). In the second phase, nuclear fragments and organelles condense and are ultimately packaged in membrane-bound vesicles, exocytosed and ingested by surrounding cells. In the final phase, membrane integrity is finally lost and permeability to dyes (i.e. trypan blue) occurs. The absence of inflammation differentiates apoptosis from necrosis when phagocytized by macrophages and epithelial cells (Kam, P C A 2000).

In contrast, necrotic cell death is a pathological process caused by overwhelming noxious stimuli (Lennon, S V 1991). Synchronously occurring in multiple cells, it is characterized by cell swelling, or “oncosis,” resulting in cytoplasmic and nuclear swelling and an early loss of membrane integrity. Bleb formation (blister-like, fluid filled structures) of the plasma membrane occurs, in which ultimate rupture may occur causing an influx of neutrophils and macrophages in the surrounding tissue, and leading to generalized inflammation (Majno, G 1995).

Four main groups of stimuli for apoptosis have been reported; ionizing radiation and alkylating anticancer drugs causing DNA damage, receptor mechanism modulation (i.e. glucocorticoids, tumor necrosis factor-α, nerve growth factor or Interleukin-3), enhancers of apoptotic pathways (i.e. phosphatases and kinase inhibitors), and agents that cause direct cell membrane damage and include heat, ultraviolet light and oxidizing agents (i.e. superoxide anions, hydroxyl radicals and hydrogen peroxide) (Kam, P C A 2000).

In addition to the oxidizing agents, many chemical and physical treatments capable of inducing apoptosis are also known to evoke oxidative stress (Buttke, M 1994, Chandra, J 2000). Ionizing and ultraviolet radiation both generate reactive oxygen intermediates (ROI) such as hydrogen peroxide and hydroxyl free radicals. Low doses of hydrogen peroxide (10-100 μM) induce apoptosis in a number of cell types directly establishing oxidative stress as a mediator of apoptosis. However, high doses of this oxidant induce necrosis, consistent with the concept that the severity of the insult determines the form of cell death (apoptosis vs. necrosis) that occurs. A free radical is not a prerequisite for inducing apoptosis; doxorubucin, cisplatin and ether-linked lipids are anti-neoplastics that induce apoptosis and oxidative damage.

Alternatively, oxidative stress can be induced by decreasing the ability of a cell to scavenge or quench reactive oxygen intermediates (ROI) (Buttke, M 1994). Drugs (i.e. butathionine sulfoxamine) that reduce intracellular glutathione (GSH) render cells more susceptible to oxidative stress-induced apoptosis. Cell studies report a direct relationship between extracellular catalase levels and sensitivity to hydrogen peroxide-induced apoptosis. Apoptosis induced through tumor necrosis factor-α stimulation has been demonstrated to be associated with an increase in intracellular ROI. However, this apoptosis has been inhibited by the addition of a number of antioxidants, such as thioredoxin, a free radical scavenger, and N-acetylcysteine, an antioxidant and GSH precursor.

There is growing evidence that apoptotic neutrophils have an active role to play in the regulation and resolution of inflammation following phagocytosis by macrophages and dendritic cells. A hallmark of phagocytic removal of necrotic neutrophils by macrophages is an inflammatory response including the release of proinflammatory cytokines (Vignola, A M 1998, Beutler, B 1988, Moss, S T 2000, Fadok V A, 2001). In contrast, apoptotic neutrophil clearance is not accompanied by an inflammatory response. Phagocytosis of these apoptotic cells has been shown to inhibit macrophage production of pro-inflammatory cytokines (GM-CSF, IL-β, IL-8, TNF-α, TxB2, and LTC4) with a concomitant activation of anti-inflammatory cytokine production (TGF-β, PGE2 and PAF)(Fadok, V A. 1988, Cvetanovic, M 2004). This phenomenon of suppression of proinflammatory cytokine production by macrophages has been extended to include phagocytosis of apoptotic lymphocytes (Fadok, V A 2001).

In addition to macrophages, down regulation of pro-inflammatory cytokine release in response to apoptotic cells has also been demonstrated by non-phagocytizing cells including human fibroblasts, smooth muscle, vascular endothelial, neuronal and mammary epithelial cells (Fadok, V A 1988, 2000; McDonald, P P 1999, Cvetanovic M, 2006). Apoptotic neutrophils in contact with activated monocytes elicit an immunosuppressive cytokine response, with enhanced IL-10 and TGF-β production and only minimal TNF-β and IL-1β cytokine production (Byrne, A 2002). Byrne et al. concluded that the interaction between activated monocytes and apoptotic neutrophils may create a unique response, which changes an activated monocyte from being a promoter of the inflammatory cascade into a cell primed to deactivate itself and other cellular targets.

Techniques to identify and quantify apoptosis, and distinguish this event from necrosis, may include staining with nuclear stains allowing visualization of nuclear chromatin clumping (i.e. Hoeschst 33258 and acridine orange) (Earnshaw, W C 1995). Accurate identification of apoptosis is achieved with methods that specifically target the characteristic DNA cleavages. Agarose gel electrophoresis of extracted DNA fragments yields a characteristic ‘ladder’ pattern which can be used as a marker for apoptosis (Bortner, C D 1995). A lesser extent of DNA degradation produces hexameric structures called ‘rosettes’ where necrotic cells leave a nondescript smear (Pritchard, D M 1996). Terminal transferase deoxyuridine nick-end labeling of DNA break points (TUNEL analysis), which labels uridine residues of the nuclear DNA fragments, can also be used to quantify apoptosis (Gavrieli, Y 1992).

Several signature events in the process of apoptosis may also be quantified by flow cytometry. These include dissipation of the mitochondrial membrane potential which is an early apoptotic event, externalization of phosphotidylserine through capture with annexin V, loss of plasma membrane integrity and nuclear chromatin condensation (distinguishing live, apoptotic and necrotic cells), and activation of caspase enzymes (early stage feature of apoptosis)(Technical Bulletin—InVitrogen 2004).

Vascular endothelial cells, including human umbilical vein endothelial cells (HUVECs), are known to release potent vasodilators, including nitric oxide (NO) and prostacyclins. Treatment of HUVECs with ozonated serum, an oxidative stressor, results in a significant and steady increase in NO production. Moreover, during twenty-four (24) hour HUVEC incubation with ozonated serum, inhibition of E-selectin release (a proinflammatory mediator) and no effect on endothelin-1 production (a potent vasoconstrictor) has been reported (Valacchi, G 2000). Valacchi et al. has suggested that reinfusion of ozonated blood into patients, by enhancing release of NO, may induce vasodilation in ischemic areas and reduce hypoxia.

CRP is a product of inflammation the synthesis of which by the liver is stimulated by cytokines in response to an inflammatory stimulus. CRP activates the classic complement pathway and participates in the opsonization of ligands for phagocytosis. Initially suggested as solely a biomarker and powerful predictor of cardiovascular risk, CRP now appears to be a mediator of atherogenesis. CRP has a direct effect on promoting atherosclerotic processes and endothelial cell activation. CRP potently down regulates endothelial nitric oxide synthase (eNOS) transcription and destabilizes eNOS mRNA, which decreases both basal and stimulated nitric oxide (NO) release.

In a synchronous fashion, CRP has been shown to stimulate endothelin-1 (potent vasoconstrictor) and interleukin-6 release (proinflammatory cytokine), upregulate adhesion molecules, and stimulate monocyte chemotactic protein-1 while facilitating macrophage LDL uptake. More recently, CRP has been shown to facilitate endothelial cell apoptosis and inhibit angiogenesis, as well as potentially upregulate nuclear factor kappa-B, a key nuclear factor that facilitates the transcription of numerous pro-atherosclerotic genes. The direct pro-atherogenic effects of CRP extend beyond the endothelium to the vascular smooth muscle, where it directly upregulates angiotensin type 1 receptors and stimulates vascular smooth muscle migration, proliferation, neointimal formation and reactive oxygen species production.

CRP has several deleterious effects (e.g. reduced survival, differentiation, function, apoptosis, and endothelial progenitor cell-eNOS mRNA expression) on endothelial progenitor cells which are important in neovascularization including induction of blood flow recovery in ischemic limbs and increase in myocardial viability after infarction.

### Inflammatory Diseases, Disorders and Conditions

Autoimmune/Alloimmune Diseases: Autoimmune diseases are generally believed to be caused by the failure of the immune system to discriminate between antigens of foreign invading organisms (non-self) and tissues native to its own body (self). When this failure to discriminate between self and non-self occurs and the immune system reacts against self antigens, an autoimmune disorder may arise. Autoimmune diseases, or diseases having an autoimmune component include rheumatoid arthritis, multiple sclerosis, systemic lupus erythromatosis (SLE), scleroderma, diabetes, inflammatory bowel disease, psoriasis, pemphigus, atherosclerosis (wherein the vasculature is regarded as a specific organ) and chronic heart failure.

Rheumatoid arthritis is an example of a common human autoimmune disease, affecting about 1% of the population. This disease is characterized by chronic inflammation of the synovial joints which may lead to progressive destruction of cartilage and bone.

Pemphigus is a group of autoimmune diseases characterized by the formation of watery blisters on the skin. It is an intraepidermal blistering disease characterized clinically by superficial blisters and erosions of the skin and/or mucous membranes, especially the mouth. Anti-inflammatory agents such as corticosteroids are frequently used to inhibit the inflammatory process by inhibiting specific cytokine production.

Systemic lupus erythromatosis (SLE) is an inflammation of the connective tissues and can afflict every organ system. Ninety percent of patients experience joint inflammation similar to rheumatoid arthritis. Treatment includes anti-inflammatory drugs to control arthritic symptoms and topical corticosteroids for skin. Oral steroids, such as prednisone, are used for treatment of systemic symptoms.

Scleroderma is a symptom of a group of diseases that involve the abnormal growth of connective tissue, which supports the skin and internal organs. The rheumatic component of scleroderma is characterized by inflammation and/or pain in the muscles, joints, or fibrous tissue.

Diabetes has been increasingly recognized as a disease with low-grade systemic inflammation. This mild inflammatory state is closely related to obesity and insulin resistance wherein adipocytes, especially in the obese, secrete a number of pro-inflammatory cytokines.

Psoriasis is the result of highly reactive early cellular inflammation. Psoriasis simultaneously has a rapidly proliferating epidermis, a vigorous acute inflammatory reaction, an accelerated rate of dermal breakdown and repair, and vascular and fibroblast proliferation.

Atherosclerosis involves an ongoing inflammatory response, which has a fundamental role in mediating all stages of the disease from initiation through progression and, ultimately, the thrombotic complications of atherosclerosis. Elevation in markers of inflammation predicts outcomes of patients with acute coronary syndromes. Low-grade chronic inflammation, as indicated by levels of the inflammatory marker C-reactive protein, prospectively defines risk of atherosclerotic complications.

Chronic heart failure is a debilitating condition in which the heart's ability to function as a pump is impaired, most frequently as a result of coronary artery disease or hypertension. Chronic inflammation is recognized as contributing to the development and progression of heart failure. Patients with heart failure experience a continuing decline in their health, resulting in an increased frequency of hospitalization and premature death. It is estimated that there are more than 10 million people with chronic heart failure in North America and Europe. The average five-year survival rate for all patients with heart failure is approximately 50%. In the United States alone, there are approximately 300,000 deaths associated with chronic heart failure each year.

Inflammatory bowel disease describes two autoimmune disorders of the small intestine; Crohn's disease and ulcerative colitis. Treatment includes the use of anti-inflammatory drugs, including corticosteroids for acute episodes of these diseases.

Alloimmune diseases are referred to herein as disorders such as graft versus host disease and tissue transplant rejection, in which an immune response against or by foreign, transplanted tissue can lead to serious complications or can be fatal. In the treatment of these disorders, it is desired to prevent the body from reacting against non-self antigens. Corticosteroids are frequently used to decrease inflammation by suppressing migration of polymorphonuclear leukocytes and reversing increased capillary permeability.

Neurological disorders: Inflammatory cytokines are implicated in inflammation-related disorders of the brain, namely the neuroinflammatory, neurodegenerative and neurological disorders such as Alzheimer's disease, senile dementia, multiple sclerosis, depression, Down's syndrome, Huntington's disease, peripheral neuropathies, spinal cord diseases, neuropathic joint diseases, chronic inflammatory demyelinating disease (CIPD), neuropathies including mononeuropathy, polyneuropathy, symmetrical distal sensory neuropathy, cystic fibrosis, neuromuscular junction disorders, myasthenias and Parkinson's disease.

Certain neurological brain disorders such as Down's syndrome, epilepsy, brain trauma and Huntington's disease (chorea) are currently understood to involve inflammation of brain cells as a significant component of the underlying pathology of the disorder.

Other neurological disorders which have a significant inflammatory component include Guillain-Barr syndrome (GBS), chronic inflammatory demyelinating polyneuropathy (CIDP), myasthenia gravis (MG), dermatomyositis, polymyositis, inclusion body myositis, ischemic stroke, neurosarcoidosis, vascular dementia, vasospasm, subarachnoid hemorrhage, adrenal leukocytic dystrophy (storage disorders), inclusion body dermatomyostis, minimal cognitive impairment and Duchenne muscular dystrophy.

Chronic inflammatory demyelinating polyneuropathy (CIDP) is a neurological disorder characterized by slowly progressive weakness and sensory dysfunction of the legs and arms. The disorder, which is sometimes called chronic relapsing polyneuropathy, is caused by damage to the myelin sheath of the peripheral nerves. Primary symptoms include slowly progressive muscle weakness and sensory dysfunction affecting the upper and lower extremities.

CIDP is closely related to the more common, acute demyelinating neuropathy known as Guillain-Barr syndrome (GBS). CIPD is considered the chronic counterpart of the acute disease GBS. CIDP is distinguished from GBS, chiefly by clinical course and prognosis.

Guillain-Barr Syndrome (GBS) is an acute predominately motor polyneuropathy with spontaneous recovery that may lead to severe quadriparesis and requires artificial ventilation in 20-30% of patients. The most common disease that underlies this syndrome has been classified as acute inflammatory demyelinating polyneuropathy (AIDP).

Autoimmune myasthenia gravis (MG) is a disorder of neuromuscular transmission leading to fluctuating weakness and abnormal fatigueability. Weakness is attributed to the blockade of acetylcholine receptors at the neuromuscular endplate by circulating autoantibodies, followed by local complement activation and destruction of acetylcholine receptors.

The causes of the inflammatory muscle diseases dermatomyositis, polymyositis and inclusion body myositis (IBM) are unknown, but immune mechanisms are strongly implicated. Although clinically and immunopathologically distinct, these diseases share three dominant histological features: inflammation, fibrosis and loss of muscle fibers.

Sarcoidosis is a multisystem chronic disorder with unknown cause and a worldwide distribution. Neurosarcoidosis is a complication of sarcoidosis involving inflammation and abnormal deposits in the tissues of the nervous system. Sudden, transient facial palsy is common with involvement of cranial nerve VII. Other manifestations include aseptic meningitis, hydrocephalus, parenchymatous disease of the central nervous system, peripheral neuropathy and myopathy. Intracranial sarcoid may mimic various forms of meningitis, including carcinomatous and intracranial mass lesions such as meningioma, lymphoma and glioma, based on neuroradiological imaging.

Vascular dementia (VaD) is the general term for dementia caused by organic lesions of vascular origin, such as cerebral infarction, intracerebral hemorrhage or ischemic changes in subcortical white matter. It is the most frequent cause of dementia after Alzheimer's dementia accounting for about 20% of cases and 50% in subjects over 80 years. An inflammatory component has been indicated in a variety of underlying diseases under the umbrella of VaD.

Cerebral vasospasm is delayed onset cerebral artery narrowing in response to blood clots left in the subarachnoid space after spontaneous aneurysmal subarachnoid hemorrhage (SAH). It is angiographically characterized as the persistent luminal narrowing of the major extraparenchymal cerebral arteries and affects the cerebral microcirculation and causes decreased cerebral blood flow (CBF) and delayed ischemic neurological deficits. Production of pro-inflammatory cytokines in the cerebrospinal fluid following SAH has also been demonstrated.

Duchenne muscular dystrophy (DMD) is one of the most common, inherited, lethal disorders in childhood. It is an X-linked neuromuscular disease that affects 1 in 3500 males. Progressive muscle weakness begins between 2 and 5 years of age and ultimately leads to premature death by respiratory or cardiac failure during the middle to late twenties. DMD patients lack the protein dystrophin which is an essential link in the complex of proteins that connect the cytoskeleton to the extracellular matrix. Evidence suggests that these patients exhibit immune cells similar to those found in inflammatory disease such as polymyositis. Current research further indicates that T cells may play a role in the pathology of dystrophin deficiency as well as an autoimmune component.

Multiple sclerosis, an autoimmune disease of the central nervous system expresses an inflammatory component that is treated with corticosteroids to reduce inflammation.

Ischemic stroke is caused by a blockage in a blood vessel that stops the flow of blood and deprives the surrounding brain tissue of oxygen. Within seconds to minutes of the loss of perfusion to a portion of the brain, an ischemic cascade is initiated. Allowed to progress, it will cause a central area of irreversible infarction surrounded by an area of potentially reversible ischemic penumbra. Metabolic aberrations create an intracellular gradient responsible for intracellular accumulation of water (i.e. cytotoxic edema). This is followed by the formation of pro-inflammatory cytokines and other factors that, in turn, cause further inflammation and microcirculatory compromise resulting in vasogenic edema. In addition, there is evidence indicating that the vascular endothelium plays a major role in the regulation of blood flow and is of importance in connection with cardiovascular disorders including inflammatory diseases. A dysfunctional endothelium may be a contributory factor in the demise of the ischemic penumbra.

Edema is a condition characterized by abnormally large fluid volume in the circulatory system or in tissues between the body's cells (interstitial spaces) which can cause mild to severe swelling in one or more parts of the body. Factors that can upset the balance of fluid in the body to cause edema, including: immobility of the lower limbs, medications (steroids, hormone replacements, non-steroidal anti-inflammatory drugs (NSAIDs), intake of salt, menstruation and pregnancy. Medical conditions that may cause edema include: heart failure, kidney disease, thyroid or liver disease, malnutrition, thrombosis, infection, lymphedema and solid tumors. Symptoms vary depending on the cause of edema. In general, weight gain, puffy eyelids, and swelling of the legs may occur as a result of excess fluid volume. Pulse rate and blood pressure may be elevated.

Edema-related conditions include traumatic brain injury, which is associated with a variety of physiological and cellular phenomena such as ischemia, increased permeability of the blood-brain barrier, necrosis and motor and memory dysfunction. Ischemia caused by the initial brain injury induces a cascade of secondary events which ultimately lead to cellular death. Experimental models for closed head injury have demonstrated induction of pro-inflammatory cytokine release which in conjunction with damage to endothelial cells results in disruption of the blood brain barrier integrity.

Spinal cord injury initiates a cascade of biochemical and cellular events that includes an inflammatory immune system response. Immediately after the injury, a major reduction in blood flow to the site occurs. Cells that line the still-intact blood vessels in the spinal cord begin to swell, which continues to reduce blood flow to the injured area. Influx of fluid and immune cells (neutrophils, T cells, macrophages and monocytes) past the compromised blood brain barriers causes inflammation, which is exacerbated by pro-inflammatory cytokine release by a variety of neuroglial cells and astrocytes furthering damage to the injured spinal cord.

Soft tissue injury is an acute connective tissue injury that may involve muscle, ligament, tendon, capsular and cartilaginous structures. In a sprain, strain, bruise or crush, the local network of blood vessels is damaged, and the oxygenated blood can no longer reach the affected tissue, resulting in cellular damage. Increases in temperature, redness, pain and swelling (localized edema) characterize the initial inflammatory phase. Inflammatory swelling starts to develop approximately two hours after the injury and may last for days or weeks. Immediate management includes control of the acute inflammatory response.

A variety of imaging techniques are available to assess the degree of edema surrounding an infarct site and blood flow to the ischemic penumbra in ischemic brain stroke patients. Examples include: Computerized Axial Tomography (CT scan), Doppler sonography, and Magnetic Resonance Imaging

At present, the most common method of assessing endothelium-mediated vasorelaxation is brachial arterial (BA) imaging, which involves taking high resolution ultrasound images to determine the diameter of the BA before and after several minutes of arterial occlusion. The change in arterial diameter is a measure of flow-mediated vasorelaxation (FMVR).

Other methods of vasorelaxation measurement include inducing an artificial pulse at the superficial radial artery via a linear actuator. An ultrasonic Doppler stethoscope detects the pulse 10-20 cm upstream from the initial pulse. The delay between pulse application and detection provides the pulse transit time (PTT). PTT is measured before and after five minutes of BA occlusion and reactive hyperemia. As the blood flow increases after occlusion, the endothelial cells that line the inner wall of the artery sense the increased friction and chemical composition of the blood and release relaxing agents into the artery's smooth muscle. The healthier the vascular system, the better the endothelial layer functions and the greater the difference will be between the pre- and post-occlusion measurements.

Measures of patient inflammation may include physical assessment of joint stiffness, elevated temperature and reported pain. Laboratory measures of inflammation may include elevation in leukocyte count including differential, coagulation system measurement, inflammatory cytokine (including IL-6 and IL-8) elevation, and increases in C-reactive protein (including high sensitivity CRP) and procalcitonin levels.

Historically, ozone has been used as a disinfectant or sterilizing agent in a wide variety of applications. These include fluid-based technologies such as purification of potable water, sterilization of fluids in the semi-conductor industry, disinfection of wastewater and sewage, and inactivation of pathogens in biological fluids. Ozone has also been used in the past as a topical medicinal treatment, as a systemic therapeutic and as a treatment of various fluids that were subsequently used to treat a variety of diseases. Specifically, there have been numerous attempts utilizing a variety of ozone-based technologies to treat inflammatory diseases in patients.

Previous technologies were incapable of measuring and differentiating between the amount of ozone that was delivered and the amount of ozone actually absorbed and utilized. This meant previous medicinal technologies for use in patients were incapable of measuring, reporting or differentiating the amount of ozone delivered from the amount that was actually absorbed and utilized. This problem made regulatory approval as a therapeutic unlikely. In the treatment of inflammatory diseases, previous technologies were also incapable of measuring, reporting or differentiating the amount of ozone delivered from the amount that was actually absorbed by the fluid and utilized by the patient. The inability to measure the amount of ozone absorbed may result in excessive absorption resulting in unacceptable levels of cellular necrosis in the leukocyte fraction of the treated blood, which when reinfused may result in promotion of an inflammatory response. Furthermore, any technology considered to treat inflammatory diseases utilizing blood ex vivo with ozone may have to be able to maintain the biological integrity of the fluid for its subsequent intended therapeutic use.

In addition, early approaches of mixing ozone with fluids employed gas-fluid contacting devices that were engineered with poor mass transfer efficiency of gas to fluids. Later, more efficient gas-fluid contacting devices were developed, but these devices used construction materials that were not ozone inert and therefore, reacted and absorbed ozone. This resulted in absorption of ozone by the construction materials making it impossible to determine the amount of ozone delivered to and absorbed by the fluid. Furthermore, ozone absorption by construction materials likely caused oxidation and the subsequent release of contaminants or deleterious byproducts of oxidation into the fluid.

Experimental research confirms the problem of ozone absorption by construction materials. An ozone/oxygen admixture at 1200 ppmv was passaged through a commercially available membrane oxygenator. For a period in excess of two hours, a majority of the ozone delivered to the device was absorbed by the construction materials. This data strongly suggests commercially available membrane gas-fluid contacting devices, made from ozone reactive materials, cannot be used with ozone, and supports the necessity to develop novel ozone-inert gas-fluid contacting devices.

In addition, prior methods do not quantify the amount of ozone that does not react with the biological fluid. The inability to measure residual-ozone has led to inaccurate and imprecise determinations of both the amount of ozone delivered to the fluid, and the amount of ozone actually absorbed and utilized by the fluid.

Prior technologies also include inefficient methods to mix ozone with fluids yielding irregular exposure. For example, relatively large amounts of ozone may be exposed to some of the fluid and less to other portions. The result of this inefficient mixing causes a wide variation in the amount of ozone exposed to the fluid. This wide variation in ozone exposure may cause diverse biochemical events including unacceptable levels of cellular necrosis in various portions of the fluid leading to untoward and irreproducible results.

Prior techniques also failed to recognize that fluids of varying composition display different absorption phenomena. Specifically, the range of values for extracellular antioxidants in blood, including ascorbic acid (0.4-1.5 mg/dL), uric acid (2.1-8.5 mg/dL), bilirubin (0-1.0 mg/dL) and Vitamin A (30-65 μg/dL) and other oxidizable substrates, including cholesterol (140-240 mg/dL), LDL-cholesterol (100-159 mg/dL), HDL-cholesterol (33-83 mg/dL) and triglycerides (45-200 mg/dL), may alter the amount of ozone necessary to be delivered to the fluid, and subsequently absorbed and utilized to achieve a desired clinical effect.

### BRIEF SUMMARY OF THE INVENTION

In accordance with the present invention, methods for therapeutic treatment of inflammatory conditions in a mammalian subject are provide which provide clinical benefits, including reduction of inflammation, vasorelaxation, reduction in edema and increased blood flow. Methods of the invention generally comprise extracorporeal treatment of blood, blood fractionate, or other biological by exposure of such fluids to a precise, measured amount of ozone to produce a treated fluid that has a quantifiable absorbed dose of ozone. Upon reinfusion of the fluid having the quantified absorbed dose of ozone into the subject, a number of biochemical events result, such as the induction of apoptosis in the leukocyte fraction, thereby providing beneficial and therapeutic effects to the subject, including but not limited to anti-inflammatory and vasorelaxatory effects beneficial in the treatment of inflammatory disorders. The method may also result in the reduction in C-reactive protein (CRP) sufficient to elicit clinical benefit, such as reduction of inflammation and increased blood flow through vasodilation and/or neovascularization.

The methods of the invention further include reinfusion of the treated fluid having the quantified absorbed dose of ozone into a mammalian subject to provide and elicit therapeutic effects which treat the disease, condition or symptoms of the disclosed diseases, as well as other actual or potential diseases.

The methods of the present invention further provide for the manufacture of substances or compositions that are useful in the therapeutic treatment of inflammatory disease, and related symptoms and conditions of these diseases. The methods of the present invention further provide for the use of such substances and compositions in the manufacture of medicaments or other administrable substances for the therapeutic treatment of inflammatory disease and related symptoms and conditions of these diseases.

The methods of the invention provide therapeutic treatments for any disease or condition in which inflammation is a component in a mammalian patient. Inflammatory diseases and conditions may include rheumatoid arthritis, multiple sclerosis, systemic lupus erythromatosis (SLE), scleroderma, diabetes, inflammatory bowel disease, psoriasis, pemphigus, atherosclerosis, chronic heart failure, graft versus host reaction and tissue transplant rejection.

The methods of the present invention also provide for the prophylactic or therapeutic treatment of neurological brain diseases or disorders which include an inflammatory component, and may include Alzheimer's disease, ischemic brain stroke, senile dementia, multiple sclerosis, depression, Down's syndrome, Huntington's disease, peripheral neuropathies, spinal cord diseases, neuropathic joint diseases, chronic inflammatory demyelinating disease (CIPD), neuropathies, including mononeuropathy, polyneuropathy, symmetrical distal sensory neuropathy, cystic fibrosis, neuromuscular junction disorders, myasthenias and Parkinson's disease.

The methods of the present invention also provide a therapeutic approach to reduction of inflammation and vasorelaxation, which may result in a reduction in edema and improvement in impaired blood flow to an inflamed area and may include traumatic brain injury, spinal cord injury and soft tissue injuries in a mammalian patient.

The methods of the present invention comprise subjecting an amount of blood, blood fractionate, or other biological fluid, extracorporeally, to an amount of ozone delivered by an ozone delivery system, resulting in the absorption of a quantifiable absorbed-dose of ozone by the biological fluid. The treated fluid is then reintroduced or reinfused autologously to the subject. The method may also provide for the maintenance of the biological integrity of the treated biological fluid.

The method employs an ozone-delivery system for delivering and manufacturing a measured amount of an ozone/oxygen admixture, which is able to measure, control and report and differentiate between delivered-ozone and the quantifiable absorbed-dose of ozone. The system may include improved gas-fluid contacting devices that maximize gas-fluid mass transfer. All gas contact surfaces of the system, including one or more gas-fluid contacting devices and all pathways transporting an ozone or an ozone/oxygen admixture to and from the gas-fluid contacting device, are made from ozone-inert construction materials that do not absorb ozone and do not introduce contaminants or deleterious byproducts of oxidation into a fluid.

Embodiments of methods of the present invention further comprise extracorporeally subjecting an aliquot of a mammalian patient's blood, or the separated cellular fractions of the blood, or mixtures of the separated cells, including platelets, to a measured amount of ozone such that the aliquot of fluid absorbs a quantifiable absorbed-dose of ozone. On re-introduction of this autologous aliquot to the patient, the treated blood, blood fractionate, or other biological fluid with a quantifiable absorbed-dose of ozone provides certain clinically beneficial effects to the patient. These effects result in the improvement in inflammatory disorder-related conditions including reduction of inflammation, relaxation of the vascular endothelium, reduction in edema and increased blood flow.

The methods of the present invention comprise treatment of blood or fractionates thereof to cause sufficient leukocyte apoptosis necessary to elicit clinical benefit when the treated fluid is reinfused autologously into a patient. The promotion of leukocyte apoptosis is achieved without excessive necrosis necessary to elicit clinical benefit when reinfused autologously into a patient.

Certain embodiments of the invention comprise the method of connecting a subject to a device for withdrawing blood or other biological fluid, withdrawing blood or biological fluid from the subject, delivering a measured amount of ozone to the blood or biological fluid under conditions which may maintain the biological integrity of the blood or biological fluid, and subsequently reinfusing the treated fluid into the subject.

The methods of the present invention further comprise measurement or evaluation of the efficacy of clinical benefits described herein by use of a variety of diagnostic tools to measure, for example, reduction in joint stiffness, reduction in temperature and reported pain, normalization of leukocyte count including differential, coagulation system measurement, inflammatory cytokines, C-reactive protein (including high sensitivity CRP) and procalcitonin levels.

The methods of the present invention may further include therapeutic treatments that reduce inflammation by the reduction in pro-inflammatory cytokines (e.g. interferon-gamma, TNF-gamma, IL-6, IL-8 and IL-12) and/or an increase in anti-inflammatory cytokines (e.g. interleukin-4 and IL-10) released by immunomodulatory cells. The effect of reducing inflammation may result in any number of clinical benefits including improvement in blood flow yielding enhanced oxygenation.

One objective of the present method provides for treatment of inflammatory diseases including a method of delivery of a measured amount of ozone and subsequent absorption of a quantifiable absorbed-dose of ozone by blood, blood fractionate, or other biological fluid extracorporeally, which when reinfused autologously into a patient may cause a reduction in CRP sufficient to elicit clinical benefit.

The methods of the present invention are directed to therapeutic treatments which reduce inflammation and thereby relax the vascular endothelium to provide clinically beneficial effects to the patient in the treatment of inflammatory disorders. The therapeutic treatments of the present invention to reduce inflammation also reduce edema, thereby providing clinical benefits in the treatment of inflammatory disorders.

The methods of the present invention provide therapeutic treatments which induce apoptosis in the leukocyte fraction of blood or blood fractionate to elicit a reduction of inflammation when reinfused autologously into a patient, and to induce such apoptosis without causing excessive necrosis. The evaluation of the efficacy and/or sufficiency of the induced leukocyte apoptosis may be evaluated, in accordance with further processes of the present invention, by a number of diagnostic methods including light microscopy with nuclear stains, electrophoretic analysis of DNA fragmentation, TUNEL analysis and multiparameter flow cytometry.

The methods of the present invention also provide therapeutic treatments which are effective to increase blood flow in patients suffering from inflammatory disorders. The evaluation of the efficacious inducement of increased blood flow due to the therapeutic methods of the present invention may be evaluated, in accordance with the methods of the present invention, by a variety of diagnostic tools including MRI and Doppler imaging techniques.

The methods of the present invention also provide therapeutic treatments which are effective to reduce edema in patients suffering from inflammatory disorders. The evaluation of the efficacy of reduction of edema due to the therapeutic treatment methods of the present invention may be evaluated, in accordance with the methods of the present invention, by a variety of diagnostic tools including MRI, CT and Doppler imaging techniques. In accordance with the present invention, the reduction in edema may be effected by the therapeutic treatment of a patient's blood, blood fractionate or other biological fluid to induce leukocyte apoptosis, without excessive necrosis, as a means of reducing edema.

The methods of the present invention are effective in reducing edema which may, in turn, increase blood flow and be clinically beneficial in the treatment of inflammatory disorders. The methods are also directed to effecting a relaxation of the vascular endothelium in patients suffering from inflammatory disorders. The present methods effect relaxation in the vascular endothelium in patients suffering from inflammatory disorders which may be the result of vasodilation, and involve release of nitric oxide and prostacyclins leading to improvement in endothelial function including endothelial cellular repair or replacement.

The present methods also effect relaxation of the vascular endothelium in patients suffering from inflammatory disorders, which may be the result of vasodilation, by promoting inhibition of vasoconstrictors, thereby leading to improvement in endothelial function, including endothelial cellular repair or replacement. The effects of modifying vasodilation or inhibition of vasoconstrictors by use of the therapeutic methods may also lead to improvement in endothelial function which may be clinically beneficial in the treatment of inflammatory conditions including improvement in blood flow yielding enhanced oxygenation. These effects may be evaluated, in accordance with the methods of the present invention, by a variety of diagnostic methods including ultrasonography based flow-mediated vasorelaxation (FMVR) and pulse transit time (PTT).

In one embodiment of the invention, the therapeutic treatment of blood or other biological fluid withdrawn from a subject is carried out by a discontinuous flow method. The method comprises connecting a subject to a device for withdrawing blood, withdrawing blood, delivering a measured amount of ozone to the blood or a blood fractionate under conditions which maintain the biological integrity of the blood or blood fractionate and then reinfusing the treated blood, blood fractionate or other biological fluid into the subject, the blood, blood fractionate or other biological fluid containing a quantifiable absorbed-dose of ozone.

The discontinuous flow method may be used to treat a subject's blood or a fraction thereof, including plasma or serum, by a discontinuous flow method. The method comprises connecting a subject to a device for withdrawing blood, withdrawing blood containing cells from the subject, separating the cellular fraction from the blood, and delivering a measured amount of ozone to this fraction under conditions which maintain the biological integrity of the blood or blood fraction. The treated fraction is subsequently recombined with the acellular fluid component of the blood and is re-infused into the subject.

Further embodiments of the methods of the present invention comprise extracorporeally subjecting an aliquot of a mammalian patient's blood, or the separated cellular fractions of the blood, or mixtures of the separated cells, including platelets, to a measured amount of ozone such that the aliquot absorbs a quantifiable absorbed-dose of ozone. On re-introduction of the autologous treated aliquot to the patient, the blood or blood fractionate with a quantifiable absorbed-dose of ozone provides certain clinically beneficial effects in the treatment of inflammatory disorders including rheumatoid arthritis, multiple sclerosis, systemic lupus erythromatosis (SLE), scleroderma, diabetes, inflammatory bowel disease, psoriasis, pemphigus, atherosclerosis, chronic heart failure, graft versus host reaction and tissue transplant rejection. These effects may result in the improvement in inflammatory disorder-related conditions including reduction of inflammation, relaxation of the vascular endothelium, reduction in edema and increased blood flow.

The methods of the present invention are also directed to the therapeutic treatment of neurological brain diseases or disorders that include an inflammatory component, and may include Alzheimer's disease, ischemic brain stroke, senile dementia, multiple sclerosis, depression, Down's syndrome, Huntington's disease, peripheral neuropathies, spinal cord diseases, neuropathic joint diseases, chronic inflammatory demyelinating disease (CIPD), neuropathies including mononeuropathy, polyneuropathy, symmetrical distal sensory neuropathy, cystic fibrosis, neuromuscular junction disorders, myasthenias and Parkinson's disease by administration to the patient of such treated blood, blood fractionate, or other biological fluid in accordance with the present invention.

The methods of the present invention are further directed to providing therapeutic treatment for disease conditions that have or present an inflammatory component, including traumatic brain injury, spinal cord injury and soft tissue injuries in a mammalian patient, by administration to the patient of treated blood, blood fractionate, or other biological fluid in accordance with the methods described herein. These effects may result in the improvement in inflammatory disorder-related conditions including reduction of inflammation, relaxation of the vascular endothelium, reduction in edema, and increased blood flow.

The methods of the present invention and therapeutic treatments are further directed to providing treatment of joint tenderness and, improving paralysis, providing improvement from motor weakness, providing improvement, providing improvement in ocular and auditory functions, improvement in cognitive function and verbal communication, all in patients suffering from an inflammatory disorder. Providing therapeutic treatment for these conditions is directed to further promoting re-attainment of independence in patients suffering from an inflammatory disorder, and improving the rate of overall survival in patients suffering from an inflammatory disorder.

The methods of therapeutic treatment of the present invention further provide treatment of inflammatory diseases wherein there is a shift from a pro-inflammatory state to an anti-inflammatory state of the vascular endothelium, and provide for relaxation of the vascular endothelium through the release of anti-inflammatory cytokines including interleukin-4 and interleukin-10 and TGF-gamma. The therapeutic methods further provide for the relaxation of the vascular endothelium through the inhibition of pro-inflammatory cytokines including interferon-gamma, TNF-gamma, IL-1, IL-6, IL-8 and IL-12.

The methods of therapeutic treatment of the present invention further provide treatment of inflammatory diseases by causing the release of endothelium-derived relaxing factor, nitric oxide, prostacyclin or other related vasodilatory compounds. The methods of therapeutic treatment of the present invention further provide treatment of inflammatory diseases wherein there is an increase in blood flow to an ischemic area, including providing increased oxygen delivered to an ischemic area.

### BRIEF DESCRIPTION OF DRAWINGS

To further clarify the present invention, treatment systems of the present invention using an ozone delivery system are illustrated in the appended drawing, which schematically illustrate what is currently considered the best mode for carrying out the invention;

FIG. 1 illustrates, in a schematic diagram, alternative methods of carrying out treatment of a fluid from a patient comprising a continuous loop format and, alternatively, a discontinuous flow method.

### DETAIL DESCRIPTION OF THE PREFERRED EMBODIMENTS OF THE INVENTION

### Definitions

As used herein, “comprising,” “including,” “containing,” “characterized by,” and grammatical equivalents thereof are inclusive or open-ended terms that do not exclude additional, unrecited elements or method steps, but also include the more restrictive terms “consisting of and “consisting essentially of.”

As used herein and in the appended claims, the singular forms, for example, “a”, “an”, and “the,” include the plural, unless the context clearly dictates otherwise. For example, reference to “a gas-fluid contacting device” includes a plurality of such gas-fluid contacting devices, and reference, for example, to a “protein” is a reference to a plurality of similar proteins, and equivalents thereof.

An “ozone/oxygen admixture” refers to a concentration of ozone in an oxygen carrier gas. Various units of concentration utilized by those skilled in the art include: micrograms of ozone per milliliter of oxygen, parts (ozone) per million (oxygen) by weight (‘ppm’) and parts per million by volume (‘ppmv’). As a unit of concentration for ozone in oxygen, ppmv is defined as the molar ratio between ozone and oxygen. One ppmv of ozone is equal to 0.00214 micrograms of ozone per milliliter of oxygen. Additionally, one ppm ozone equals 0.00143 micrograms of ozone per milliliter of oxygen. In terms of percentage ozone by weight, 1% ozone equals 14.3 micrograms of ozone per milliliter of oxygen. All units of concentration and their equivalents are calculated at standard temperature and pressure (i.e. 25° C. at 1 atmosphere).

“Delivered-ozone” is the amount of ozone contained within a volume of an ozone/oxygen admixture that is delivered to a fluid, and is synonymous with the delivery of a measured amount of ozone.

“Absorbed-ozone” is the amount of delivered-ozone that is actually absorbed and utilized by an amount of fluid, and is synonymous with an absorbed dose of ozone.

“Residual-ozone” is the amount of delivered-ozone that is not absorbed such that:

Residual-ozone=delivered-ozone−absorbed-dose of ozone.

An “interface” is defined as the contact between a fluid and an ozone/oxygen admixture.

“interface-time” is the time that a fluid resides within a gas-fluid contacting device and is interfaced with an ozone/oxygen admixture.

“Interface surface area” is defined as the dimensions of the surface within a gas-fluid contacting device over which a fluid flows and contacts an ozone/oxygen admixture.

“Elapsed-time” is the time that a fluid circulates throughout an ozone delivery system, including passage through one or more gas-fluid contacting devices, connecting tubing and an optional reservoir.

“Ozone-inert materials” are defined as construction materials that do not react with ozone in a manner that introduces contaminants or deleterious byproducts of oxidation of the construction materials into a fluid, and materials that do not absorb ozone.

“Non-reactive” is defined as not readily interacting with other elements or compounds to form new chemical compounds.

“Measured-data” is defined as information collected from various measuring components (such as an inlet ozone concentration monitor, exit ozone concentration monitor, gas flow meter, fluid pump, data acquisition device, humidity sensor, temperature sensor, pressure sensor, absorbed oxygen sensor) throughout the system.

“Calculated-data” is defined as the mathematical treatment of measured-data by a data acquisition device.

“Absorption of ozone by a biological fluid” is defined as the phenomenon wherein ozone reacts with the fluid by a variety of mechanisms, including oxidation. Regardless of the mechanism involved, the reaction occurs instantaneously, and the products of this reaction may include oxidative byproducts.

A “biological fluid” is defined as a composition originating from a biological organism of any type. Examples of biological fluids include blood, blood products and other fluids, such as saliva, urine, feces, semen, milk, tissue, tissue samples, homogenized tissue samples, gelatin and any other substance having its origin in a biological organism. Biological fluids may also include synthetic materials incorporating a substance having its origin in a biological organism, such as a vaccine preparation containing alum and a virus (the virus being the substance having its origin in a biological organism), cell culture media, cell cultures, viral cultures, and other cultures derived from a biological organism.

A “blood fractionate” is defined as including any cellular (i.e. packed red blood cells, platelet concentrate) or acellular fractionate (i.e. plasma, serum, therapeutic protein compositions) derived from blood.

“In vivo” use of a material or compound is defined as the introduction of a material or compound into a living human, mammal, or vertebrate.

“In vitro” use of a material or compound is defined as the use of the material or compound outside a living human, mammal, or vertebrate, where neither the material nor compound is intended for re-introduction into a living human, mammal or vertebrate. An example of an in vitro use would be the analysis of a component of a blood sample using laboratory equipment.

“Ex vivo” use of a process is defined as using a process for treatment of a biological material such as a blood product outside of a living human, mammal, or vertebrate. For example, removing blood from a human and subjecting that blood to a method to treat an inflammatory disease is defined as an ex vivo use of that method if the blood is intended for reintroduction into that human or another human. Reintroduction of the human blood into that human or another human would be an in vivo use of the blood, as opposed to an ex vivo use of the method.

“Extracorporeal” is defined as a state wherein blood or blood fractionate is treated outside (ex vivo) of the body, for example, in the delivery of a measured amount of ozone to a sample of patient's blood.

“Synthetic media” is defined as an aqueous synthetic blood or blood product storage media.

A “pharmaceutically-acceptable carrier” or “pharmaceutically-acceptable vehicle” is defined as any liquid including water, saline, a gel, salve, solvent, diluent, fluid ointment base, liposome, micelle or giant micelle, which is suitable for use in contact with a living animal or human tissue without causing adverse physiological responses, and which does not interact with the other components of the composition in a deleterious manner.

“Biologically active” is defined as capable of effecting a change in the living organism or component thereof.

The “biological integrity of a biological fluid” is a quality or state of a fluid that, subsequent to the method of treating for inflammatory diseases or related conditions and symptoms described herein, sufficiently maintains its functionality upon re-infusion into a mammalian patient.

“Inflammatory diseases” are defined as diseases that are characterized by activation of the immune system to abnormal levels characterized by inflamed tissue, characterized by pain, swelling, redness and heat.

“Neurodegenerative diseases” are defined as disorders caused by the deterioration of certain nerve cells causing them to function abnormally, eventually bringing about their death.

“Autoimmune diseases” are defined as diseases believed to be caused by the failure of the immune system to discriminate between antigens of foreign invading organisms (non-self) and tissues native to its own body (self).

“Alloimmune diseases” are defined as diseases that result from an immune response against or by foreign, transplanted tissue.

“Edema” is defined as a condition of abnormally large fluid volume in the circulatory system or in tissues between the body's cells (interstitial spaces).

“C-reactive protein” is defined as a liver-synthesized, acute phase reactant protein regarded as a marker of acute inflammation capable of activating the classical compliment pathway and opsonizing ligands for phagocytosis.

The present invention provides methods for therapeutic treatment of inflammatory diseases mediated by the delivery to an amount of blood, blood fractionate, or other biological fluid, an amount of ozone which results in the absorption of a quantifiable absorbed-dose of ozone, and reinfusion of the treated fluid into the patient, resulting in clinical benefit, which may include reduction of inflammation, relaxation of the vascular endothelium, reduction in edema and increased blood flow.

Diseases targeted as potential candidates for therapeutic treatment by the methods disclosed in the present invention include rheumatoid arthritis, multiple sclerosis, systemic lupus erythromatosis (SLE), scleroderma, diabetes, inflammatory bowel disease, psoriasis, pemphigus, atherosclerosis, chronic heart failure, graft versus host reactions including tissue transplant rejection, Alzheimer's disease, ischemic brain stroke, senile dementia, depression, Down's syndrome, Huntington's disease, peripheral neuropathies, spinal cord diseases, neuropathic joint diseases, chronic inflammatory demyelinating disease (CIPD) and neuropathies, including mononeuropathy, polyneuropathy, symmetrical distal sensory neuropathy, cystic fibrosis, neuromuscular junction disorders, myasthenias, Parkinson's disease, traumatic brain injury, spinal cord injury and soft tissue injuries.

The methods of the present invention, as described further below, generate leukocyte apoptosis without excessive necrosis, sufficient to reduce inflammation, reduce edema, improve impaired blood flow and relax the vascular endothelium once the treated blood, blood fractionate, or other biological fluid is reinfused into the patient. The methods as described below also cause or promote reduction in CRP sufficient to elicit clinical benefit.

The methods of the present invention comprise withdrawing from a mammalian patient or subject who suffers from, or is believed to suffer from, inflammatory disease and related conditions, an amount of blood or other biological fluid for treatment. The withdrawn blood or biological fluid is subjected to a measured amount of ozone from an ozone delivery system. The blood or biological fluid, which is treated extracorporeally through the use of the ozone delivery system, absorbs a quantifiable absorbed-dose of ozone. The treated fluid is subsequently reinfused into the same patient. This autologous blood or other biological fluid sample which contains a quantified absorbed-dose of ozone therapeutically effects improvement in any inflammatory disease-related condition.

In accordance with the invention, the biological fluid withdrawn from the patient may be blood, a blood fractionate or other biological fluid. The withdrawn fluid may be characterized as an aliquot of blood, a blood fractionate or other biological fluid

Specifically, the methods of the invention may comprise subjecting an aliquot of a mammalian patient's blood, or the separated cellular fractions of the blood, or mixtures of the separated cells, including platelets, to a measured amount of ozone such that the fluid absorbs a quantifiable absorbed-dose of ozone. On reintroduction of this autologous aliquot to the patient's body, the blood, blood fractionate, or other biological fluid, with a quantified absorbed-dose of ozone, provides certain clinically beneficial effects. These effects result in the improvement in inflammatory disease-related conditions, including reduction of inflammation, relaxation of the vascular endothelium, reduction in edema and increased blood flow.

In accordance with the methods of the present invention, reintroduction of treated blood, blood fractionate or other fluid autologously to a mammalian patient may be accomplished through a variety of routes, including intravenous, intramuscular and subcutaneous routes, or any combination thereof.

The therapeutic effect of blood, blood fractionate, or other biological fluid which has absorbed a quantifiable absorbed-dose of ozone, may be the induction of sufficient leukocyte apoptosis, without excessive necrosis, necessary to elicit an anti-inflammatory response when reinfused autologously into a patient. The induction of apoptosis without excessive necrosis in the leukocyte fraction of the blood, blood fractionate, or other biological fluid that has been treated in accordance with the methods described herein may be evaluated by a number of diagnostic methods including light microscopy with nuclear stains, electrophoretic analysis of DNA fragmentation, TUNEL analysis and multiparameter flow cytometry.

A therapeutic effect of blood, blood fractionate, or other biological fluid which has absorbed a quantifiable absorbed-dose of ozone, may result in the reduction of CRP when reinfused autologously into a patient and elicit clinical benefit including an anti-inflammatory response, neovascularization and vasodilation.

The effects of blood, blood fractionate or other fluid which has absorbed a quantifiable absorbed-dose of ozone, when re-infused into a mammalian patient's body may include effects that reduce edema.

In addition, the effects of blood, blood fractionate or other fluid which has absorbed a quantifiable absorbed-dose of ozone, when re-infused into a mammalian patient's body, may include effects that increase blood flow to ischemic tissue.

In accordance with the methods of the present invention, the effective promotion of reduced edema brought about in patients with inflammatory diseases may be evaluated by a variety of diagnostic tools including CT, MRI and Doppler imaging techniques. Additionally, it is a further element of the present methods to evaluate the effect of increased blood flow brought about through the methods described herein by use of a variety of diagnostic tools including MRI and Doppler imaging techniques.

The effects of blood, blood fractionate or other fluid which has absorbed a quantifiable absorbed-dose of ozone, when re-infused into a mammalian patient's body may include effects that include reduction of inflammation. Reduction of inflammation may occur though a reduction in pro-inflammatory cytokines (e.g. interferon-gamma, TNF-gamma, IL-6, IL-8 and IL-12) and/or an increase in anti-inflammatory cytokines (e.g. interleukin-4 and IL-10) released by immunomodulatory T cells. The effect of reducing inflammation may result in any number of clinical benefits including improvement in blood flow yielding enhanced oxygenation.

The effect of treated blood or blood derivative thereof with ozone by the present method to induce apoptotic leukocytes without excessive necrosis, when re-infused into a mammalian patient's body may include effects that include reduced inflammation. Reduction of inflammation may occur though a reduction in pro-inflammatory cytokines (e.g. interferon-gamma, TNF-gamma, IL-6, IL-8 and IL-12) and/or an increase in anti-inflammatory cytokines (e.g. interleukin-4 and IL-10) released by immunomodulatory cells. Reduction of inflammation may result in any number of clinical benefits including improvement in blood flow yielding enhanced oxygenation. Diagnostic markers to measure reduction of inflammation may include reduction in joint stiffness, reduction in temperature and reported pain, normalization of leukocyte count including differential, coagulation system measurement, inflammatory cytokines, C-reactive protein (including high sensitivity CRP) and procalcitonin levels.

Further therapeutic effects mediated by the methods of the invention include relaxation of vascular endothelium. This relaxation may be the result of vasodilation and involve release of nitric oxide and prostacyclins, or inhibition of vasoconstrictors leading to improvement in endothelial function including endothelial cellular repair or replacement. Vasorelaxation may be clinically beneficial in the treatment of inflammatory conditions including improvement in blood flow yielding enhanced oxygenation. Thus, in accordance with the methods of the present invention, the evaluation of an amount or degree of vasorelaxation brought about by the therapeutic treatment methods can be measured by a variety of diagnostic methods including ultrasonography based flow-mediated vasorelaxation (FMVR) and pulse transit time (PTT).

An ozone delivery system utilized in the treatment of inflammatory diseases or related symptoms and conditions delivers a measured amount of an ozone/oxygen admixture and is able to measure, control, report and differentiate between the delivered-ozone and quantifiable absorbed-dose of ozone. The system provides a controllable, measurable, accurate and reproducible amount of ozone that is delivered to a controllable, measurable, accurate and reproducible amount of a biological fluid, and controls the rate of ozone absorption by the fluid resulting in a quantifiable absorbed-dose of ozone used in the treatment of inflammatory diseases or related symptoms and conditions. The system may accomplish this by using a manufacturing component, control components, measuring components, a reporting component and calculating component (such as an ozone generator, gas flow meter, fluid pump, variable pitch platform, data acquisition device, inlet ozone concentration monitor, and exit ozone concentration monitor) that cooperate to manufacture and deliver a measured, controlled, accurate and reproducible amount of ozone, i.e., the delivered-ozone, to a fluid through the use of one or more gas-fluid contacting devices that provides for the interface between the ozone/oxygen admixture and fluid. Using control components, measuring components, a reporting component and calculating component (such as a gas flow meter, fluid pump, variable pitch platform, data acquisition device, inlet ozone concentration monitor and exit ozone concentration monitor) that cooperate, the system may instantly differentiate the delivered-ozone from the quantifiable absorbed-dose of ozone.

The system utilizes (a gas flow meter, fluid pump, variable pitch platform, data acquisition device, inlet ozone concentration monitor, and exit ozone concentration monitor) control components, measuring components, a reporting component and calculating component that cooperate and instantly report data that may include the delivered-ozone, residual-ozone, quantified absorbed-dose of ozone, interface-time, elapsed-time and the amount and flow rate of the fluid delivered to the gas-contacting device.

A particularly suitable ozone delivery system that may be used in carrying out the methods of the present invention is disclosed in U.S. Pat. No. 7,736,494 and co-pending application Ser. No. 12/813,371, the contents of which are incorporated herein in their entirety. The disclosed ozone delivery system is particularly and uniquely constructed such that all ozone-contacting surfaces of the device are made of ozone-inert material so that the amount of ozone that is actually absorbed by the biological fluid being treated is accurately determinable. That is, by virtue of being constructed with ozone-inert materials in all ozone-contacting surfaces, no ozone is absorbed by the device itself, and the determination of the amount of ozone absorbed by the biological fluid is not inaccurately reflected as a result of ozone being absorbed by any structure of the device

The ozone delivery system utilizes measuring components, reporting components and calculating components (such as an inlet ozone concentration monitor, exit ozone concentration monitor, gas flow meter, fluid pump, data acquisition device) that cooperate together to determine certain calculated-data including the delivered-ozone, the residual-ozone and the quantifiable absorbed-dose of ozone.

Delivered-ozone is an amount of ozone calculated by multiplying the measured volume of ozone/oxygen admixtures, as reported by gas flow meters, by the measured concentration of ozone within the ozone/oxygen admixture as it enters the gas-fluid contacting device, as reported by the inlet ozone concentration monitor. The measured volume of ozone/oxygen admixtures is calculated by multiplying the measured gas flow reported by gas flow meters, by the elapsed-time.

Residual-ozone is an amount of ozone calculated by multiplying the measured volume of ozone/oxygen admixtures, as reported by gas flow meters, by the measured concentration of ozone within the ozone/oxygen admixture exiting the gas-fluid contacting device, as reported by the exit ozone concentration monitor. The measured volume of ozone/oxygen admixtures is calculated by multiplying the measured gas flow reported by gas flow meters, by the elapsed-time.

The quantifiable absorbed-dose of ozone is an amount of ozone calculated by subtracting the amount of residual-ozone from the amount of delivered-ozone. The quantified absorbed-dose of ozone in the methods of the invention may range from 1 to 10,000,000 micrograms per milliliter of fluid, and may be between 1 and 10,000 ug per milliliter of fluid.

All measured-data, including measured data from the gas flow meters, inlet and exit ozone concentration monitors, the fluid pump, temperature sensors, pressure sensors, absorbed oxygen sensor and humidity sensors are reported to a data acquisition device. The data acquisition device has instant, real-time reporting, calculating and data storing capabilities to process all measured data. The data acquisition device may use any measured data or any combination of measured data as variables to produce calculated-data. Examples of calculated-data may include delivered-ozone, residual-ozone, quantified absorbed-dose of ozone, quantified absorbed-dose of ozone per unit volume of fluid, and the quantifiable absorbed-dose of ozone per unit volume of fluid per unit time.

An ozone delivery system particularly suitable to the present invention includes an ozone generator for the manufacture and control of a measured amount of an ozone/oxygen admixture and where the admixture volume contains the delivered-ozone. A commercially available ozone generator capable of producing ozone in a concentration range between 10 and 3,000,000 ppmv of ozone in an ozone/oxygen admixture may be employed. Ozone/oxygen admixture concentrations entering the gas-fluid contacting device are instantly and constantly measured in real time, through an inlet ozone concentration monitor that may utilize UV absorption as a detection methodology. A flow meter controls and measures the delivery of the delivered-ozone in an ozone/oxygen admixture to the gas-fluid contacting device at a specified admixture flow rate. Ozone/oxygen admixture flow rates are typically in the range between 0.1 and 5.0 liters per minute.

Measurement of the humidity of the ozone/oxygen admixture delivered to the gas-fluid contacting device may be included through the use of a humidity sensor. A humidity sensor port may be provided in the ozone/oxygen admixture connecting tubing; however, it can be placed in a variety of locations. For example, the humidity sensor may be located in the connecting tubing prior to the admixture's entrance into gas-fluid contacting device.

Measurement of the temperature within the gas-fluid contacting device during the interface-time may be provided by inclusion of a temperature sensor port in the gas fluid contacting device through which a temperature sensor may be inserted. The temperature at which ozone/oxygen admixtures interface fluids ranges from 4° C. to 100° C., and may be performed at ambient temperature, 25° C., for example. The temperature at which the interface occurs can be controlled by placing the gas-fluid contacting device, optional reservoir, and both gas and fluid connecting tubing in a temperature controlled environment and/or by the addition of heating or cooling elements to the gas-fluid contact device.

Measurement of the pressure within the gas-fluid contacting device during the interface-time is provided by inclusion of a pressure sensor port in the gas-fluid contacting device through which a pressure sensor may be inserted. The pressure at which an ozone/oxygen admixtures interfaces with a fluid ranges from ambient pressure to 50 psi and may be performed between ambient pressure and 3 psi, for example. A pressure sensor port may be provided in each gas-fluid contacting device to measure and report the pressure at which the interface occurs.

The concentration of the ozone/oxygen admixtures exiting the gas-fluid contacting device, and where the admixture volume contains the residual-ozone, are instantly and constantly measured in real time through an exit ozone concentration monitor that may utilize UV absorption as a detection methodology.

A fluid pump controls and measures the flow rate of the fluid delivered to the gas-fluid-contacting device at a specified fluid flow rate. Fluid flow rates through the gas-fluid contacting device typically will range from 1 ml to 100 liters per minute, and for example, may be between 1 ml to 10 liters per minute. The fluid is generally contained within a closed-loop design and may be circulated through the gas-fluid contacting device once or multiple times.

Measurement of the amount of oxygen absorbed into a fluid while it interfaces with the ozone/oxygen admixture within the gas-fluid contacting device may be provided through the use of an absorbed oxygen sensor. The sensor is inserted within the absorbed oxygen sensor port located in the tubing as it exits the gas-fluid contacting device. Measurement of absorbed oxygen may be recorded in various units, including ppm, milligrams/liter or percent saturation.

The ozone delivery system may also include a fluid access port for fluid removal. The port is generally located in the tubing member after the fluid exits through the fluid exit port of the gas-fluid contacting device and prior to an optional reservoir.

A data acquisition device, such as a DAQSTATION (Yokogawa), for example, reports, stores and monitors data instantly and in real-time, and performs various calculations and statistical operations on data acquired. Data is transmitted to the data acquisition device through data cables, including data from ozone concentration monitors, flow meters, a humidity sensor, temperature sensors, pressure sensors, a fluid pump and an absorbed oxygen sensor.

Calculated-data in carrying out the methods of the present invention include delivered-ozone, residual-ozone, and the quantifiable absorbed-dose of ozone. Measurement of the volume of the ozone/oxygen admixture delivered can be calculated though data provided from the flow meter and the time measurement capability of the data acquisition device. Measurement of the volume of fluid delivered to the gas-fluid contacting device can be calculated by the data acquisition device utilizing fluid flow rate data transmitted from the fluid pump.

The elapsed-time can be measured and controlled through the data acquisition device. The elapsed-time that the fluid circulates through the apparatus, including the gas-fluid contacting device, and is interfaced with an ozone/oxygen admixture can vary, generally for a duration of up to 120 hours. The interface-time may also be measured by the time measuring capacity of the data acquisition device. The interface-time between a fluid and an ozone/oxygen admixture may be controlled through a composite of controls. These controls include the angle of the gas fluid contacting device, the fluid flow rate via the fluid pump, and the time controlling capacity of the data acquisition device. The interface-time may vary in duration of up to 720 minutes, and generally within duration of up to 120 minutes.

Controllable variables for an ozone delivery system may include delivered amounts and concentrations of ozone in the entering ozone/oxygen admixtures, fluid flow rates, admixture flow rates, temperature in the gas-fluid contacting device, interface-time between fluid and admixture, and the elapsed-time that the fluid may circulate through the apparatus and interface with an ozone/oxygen admixture.

Measurable variables may include ozone/oxygen admixture flow rates, amounts and concentrations of ozone in the entrance and exit ozone/oxygen admixtures, fluid flow rates, temperature and pressure in the gas-contacting device, humidity of the entrance admixture to the gas-fluid contacting device, absorbed oxygen by the fluid, interface-time and elapsed-time.

Data representing controllable variables and measurable variables acquired by the apparatus allows for a variety of calculations including delivered-ozone, residual-ozone, quantifiable absorbed-dose of ozone, quantifiable absorbed-dose of ozone per unit volume of fluid and the quantifiable absorbed-dose of ozone per unit volume of fluid per unit time.

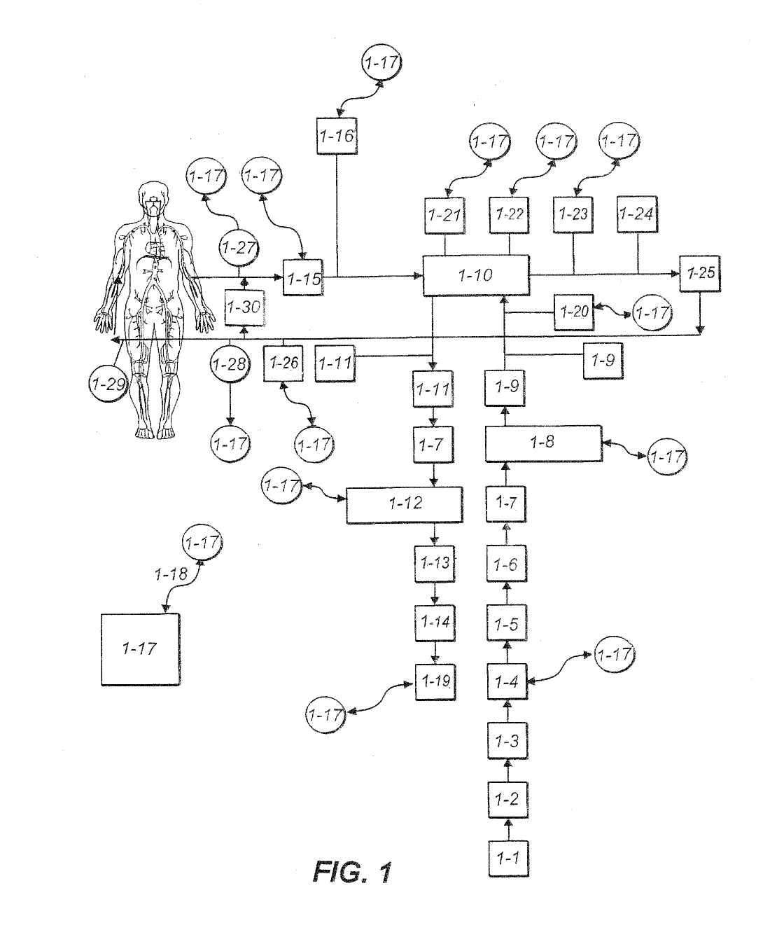

FIG. 1 schematically illustrates an embodiment of the present invention where fluid that has been taken from a subject is extracorporeally interfaced with an ozone/oxygen admixture. In general, blood may be circulated in a discontinuous manner where a fluid (e.g., an aliquot of blood) has been removed from a patient and is introduced into an ozone delivery system through a common reservoir, and is recirculated in a closed loop format. Alternatively, fluid may be circulated in a continuous loop format in a venovenous extracorporeal exchange format. As an example, this continuous loop can be established through venous access of the antecubital veins of both right and left arms. Prior to establishing a discontinuous closed loop format, blood from the patient may be anticoagulated with citrate or any other suitable anticoagulant before being introduced in to the reservoir. For an extracorporeal continuous loop circuit, a patient may optionally be anticoagulated with heparin or any other suitable anticoagulant known to those skilled in the art.

For the gas flow in either the discontinuous format or continuous loop system, oxygen flows from a pressurized cylinder (1-1), through a regulator (1-2), through a particle filter (1-3) to remove particulates, through a flow meter (1-4) where the oxygen and subsequent ozone/oxygen admixture flow rate is controlled and measured. The oxygen proceeds through a pressure release valve (1-5), through an ozone generator (1-6) where the concentration of the ozone/oxygen admixture is manufactured and controlled and where the admixture volume includes the delivered-ozone. The ozone/oxygen admixture flows through an optional moisture trap (1-7), to reduce moisture.

The admixture proceeds through an inlet ozone concentration monitor (1-8) that measures and reports the inlet ozone concentration of the ozone/oxygen admixture that contains the delivered-ozone. This real-time measurement may be based on ozone's UV absorption characteristics as a detection methodology. The ozone/oxygen admixture then passes through a set of valves (1-9) used to isolate a gas-fluid contacting device for purging of gasses. The ozone/oxygen admixture may pass an optional humidity sensor (1-20) where humidity may be measured and recorded, and into a gas-fluid contacting device (1-10) where it interfaces with fluid. The interface-time between fluid and ozone/oxygen admixture may be controlled through adjustment of a variable pitch platform, a fluid pump and the time controlling capacity of the data acquisition device.

The interface-time may then be measured by the data acquisition device (1-17). Temperature (1-21) and pressure (1-22) may be measured by the use of optional temperature and pressure sensors, respectively, inserted into their respective ports. The resultant ozone/oxygen admixture containing the residual-ozone exits the gas-fluid contacting device and flows through the exit purge valves (1-11), through a moisture trap (1-7), through an exit ozone concentration monitor (1-12), which may utilize a similar detection methodology as the inlet ozone concentration monitor (1-8), that measures and reports the exit ozone/oxygen admixture concentration. The exiting ozone/oxygen admixture then proceeds through a gas drier (1-13), through an ozone destructor (1-14) and a flow meter (1-19).

In the fluid flow for the discontinuous format, blood is introduced into the reservoir (1-30). In the continuous loop system, intravenous blood flows from the patient through tubing through a pressure gauge (1-27) which monitors the pressure of the blood flow exiting the patient. Generally, the pressure of the blood exiting the patient ranges from a negative pressure of 100-200 mm Hg, and may be between a negative pressure of 150 and 200 mm Hg, with a maximum cutoff pressure of minus 250 mm Hg. In either format, the blood flows through a fluid pump (1-15) and is optionally admixed with heparin or other suitable anticoagulant as provided by an optional heparin pump (1-16).

The blood then passes through the gas-fluid contacting device (1-10) where it interfaces with the ozone/oxygen admixture containing the delivered-ozone. Ports for the insertion of sensors may be located in the gas-fluid contacting device for the measurement of temperature and pressure, respectively. After interfacing with the ozone/oxygen admixture, the fluid exits into tubing that may contain a port for an optional absorbed oxygen sensor (1-23) followed by a fluid access port (1-24). The blood continues through an air/emboli trap (1-25) that removes any gaseous bubbles or emboli, and the blood then continues through a fluid pump (1-26).

In a discontinuous format, the blood is then directed back into the reservoir (1-30) any may continue in a recirculating mode, passaging as often as required. In the continuous loop format, the blood is directed into a pressure gauge (1-28) which monitors the pressure of the blood flow before returning the fluid to the patient. Generally, the pressure of the blood entering the patient ranges from a pressure of 100-200 mm Hg, and may be between 150 and 200 mm Hg, with a maximum cutoff pressure of 250 mm Hg. The blood continues through a priming fluid access port (1-29) that allows for the removal of the priming fluid from the extracorporeal loop. The blood is then re-infused directly into the patient.

A data acquisition device (1-17), such as a DAQSTATION (Yokogawa), for example, has time measurement capabilities, reports, stores and monitors data instantly and in real-time, and performs various calculations and statistical operations on data acquired. All data is transmitted to the data acquisition device through data cables (1-18), including: data from ozone concentration monitors (1-8) and (1-12), flow meters (1-4) and (1-19), humidity sensor (1-20), temperature sensor (1-21), pressure sensor (1-22), fluid pumps (1-15) and (1-26), pressure gauges (1-27) and (1-28), and absorbed oxygen sensor (1-23). The elapsed time, a composite of both the interface time and the period of time that the fluid circulates through the other elements of the apparatus can be measured and controlled through the data acquisition device (1-17).

Other possible configurations for an extracorporeal blood circuit known to those skilled in the art are included within the spirit of this disclosure.

One or more gas-fluid contacting devices may be included in an ozone delivery system to increase the surface area of a fluid to be treated allowing for an increase in the mass transfer efficiency of the ozone/oxygen admixture. Gas-fluid contacting devices may encompass the following properties: closed and isolated from the ambient atmosphere, gas inlet and outlet ports for the entry and exit of ozone/oxygen admixtures, fluid inlet and outlet ports for the entry and exit of a fluid, components (temperature sensor, pressure sensor and data acquisition device) for the measurement and reporting of temperature and pressure within a gas-fluid contacting device, generation of a thin film of the fluid as it flows within a gas-fluid contacting device and construction from ozone-inert construction materials including, quartz, ceramic composite, borosilicate, stainless steel, PFA and PTFE.

Gas-fluid contacting devices include designs that encompass surfaces that may be horizontal or approaching a horizontal orientation. These surfaces may include ridges, indentations, undulations, etched surfaces or any other design that results in a contour change and furthermore, may include any pattern, regular or irregular, that may disrupt the flow, disperse the flow or cause turbulence. These surfaces may or may not contain holes through which a fluid passes through. The surface of the structural elements may have the same or different pitches. Designs of gas-fluid contacting devices may include those that involve one or more of the same shaped surfaces or any combination of different surfaces, assembled in any combination of ways to be encompassed within the device which may include cones, rods, tubes, flat and semi-flat surfaces, discs and spheres.

The interface between an ozone/oxygen admixture and a fluid may be accomplished by the use of a gas-fluid contact device that generates a thin film of the fluid that interfaces with the ozone-oxygen admixture as it flows through the device. One of skill in the art will appreciate that generation of any interface that increases the surface area of the fluid and thereby maximizes the contact between a fluid and an admixture, may be used. Additional examples include the generation of an aerosol through atomization or nebulization.

The interface-time within a gas-fluid contacting device is measurable, controllable, calculable and reportable. Furthermore, the interface-time may be for duration of up to 720 minutes, generally however, for duration of up to 120 minutes. Following the interface-time, the fluid exits the gas-fluid contacting device containing the quantifiable absorbed-dose of ozone. The elapsed-time, a composite of both the interface-time and the time for circulation of a fluid through other elements of an ozone delivery system is also measurable, controllable, calculable and reportable. This elapsed-time is for duration of up to 120 hours.

The pressure at the interface between fluid and ozone/oxygen admixture within a gas-fluid contacting device may be measured. Measurement of pressure within the device may be accomplished through the use of a pressure sensor inserted at the pressure port of the gas-fluid contacting device. The pressure at which an ozone/oxygen admixture interfaces with a fluid ranges from ambient pressure to 50 psi and may be performed between ambient pressure and 3 psi.

The temperature within a gas-fluid contacting device may be controlled by housing the device such that the connecting tubing containing both gas and fluid and an optional reservoir are maintained in a controlled temperature environment. A flow hood that provides for temperature regulation is an example of a controlled temperature environment. Alternatively, the addition of heating or cooling elements to the gas-fluid contact device may provide for the control of temperature. Measurement of temperature within the device may be accomplished through the use of a temperature sensor inserted at the temperature port of a gas-fluid contacting device. The temperature at which ozone/oxygen admixtures interface fluids ranges from 4° C. to 100° C., and may be performed at ambient temperature, 25° C., for example.

Gas-fluid contacting devices may be utilized individually or in conjunction with other such devices, whether they are similar or dissimilar in construction, design or orientation. In the event that multiple devices are utilized, either of the same design, or a combination of different gas-fluid contacting devices of different designs, these devices may be arranged one after the other in succession (in series), making a single device out of multiple individual contact devices.

In a series configuration of devices, a fluid flowing through the different contact devices flows in series, from the fluid exit port of one contact device to the fluid entrance port of the next, until passing through all the devices. The ozone/oxygen admixture may flow in a number of arrangements. In one example, the ozone/oxygen admixture flows through different contact devices in series, from the admixture exit port of one contact device to the admixture entrance port of the next. As an alternative example, the ozone/oxygen admixture may flow directly from the admixture source to the entrance port of each different contact device. Another alternative is a combination of the foregoing examples where the ozone/oxygen admixture flows from the exit port of some devices to the entrance port of other devices and in addition, to the entrance of some devices directly from the admixture source. In the event that multiple devices are utilized, the resultant fluid from the terminal device can either be collected or returned to the original device and recirculated.

When arranged in series with other contact devices, interface time between the fluid and ozone/oxygen admixture is controllable, and can be adjusted based on the individual pitch chosen for each device in series, or by adding additional devices to the series. The overall interface surface area will range from 0.01 m² for an individual device, and upwards based on the number of devices serially utilized.

### Example 1

An example of data measured and calculated by the ozone delivery system that utilizes a fluid target described herein is included in Table 1. Newborn Calf Serum commercially obtained was utilized as the target fluid. A variable pitch device with variable pitch platform, as disclosed in U.S. Pat. No. 7,736,494, was employed as the gas-fluid contacting device. The following initial conditions were utilized; 300 ppmv ozone inlet concentration, 145 ml initial fluid volume, 1000 ml per minute gaseous flow rate, 189 ml per minute fluid flow rate counter current to the ozone/oxygen admixture flow. Incremental reductions in fluid volume are due to sampling of fluid through the fluid access port.

TABLE 1
NEWBORN CALF SERUM
MEASURED VARIABLES

| | | | | Average Inlet Ozone | Average Exit Ozone |
|---|---|---|---|---|---|
| Elapsed-time | Fluid Volume | Gas Flow Rate | Fluid Flow Rate | Concentration | Concentration |
| (5 min intervals) | (milliliters) | (liters/minute) | (liters/minute) | (ppmv) | (ppmv) |
| 5 | 145 | 0.998 | 0.189 | 305.2 | 38.2 |
| 10 | 143 | 0.972 | 0.189 | 361.5 | 40.4 |
| 15 | 141 | 1.000 | 0.189 | 312.7 | 20.6 |
| 20 | 139 | 1.000 | 0.189 | 314.0 | 37.3 |

CALCULATED VARIABLES

| | Average | | | | |
|---|---|---|---|---|---|
| | Differential Ozone | | | Ozone-Absorbed per | Absorbed-dose |
| Elapsed-time | Concentration | Delivered-ozone | Residual-ozone | Interval | of Ozone |
| (minutes) | (ppmv) | (ug) | (ug) | (ug) | (ug) |
| 5 | 267.0 | 3.26E+03 | 4.08E+02 | 2.86E+03 | 2.86E+03 |
| 10 | 321.1 | 7.02E+03 | 8.28E+02 | 3.34E+03 | 6.20E+03 |
| 15 | 292.1 | 1.04E+04 | 1.06E+03 | 3.12E+03 | 9.32E+03 |
| 20 | 276.7 | 1.37E+04 | 1.46E+03 | 2.96E+03 | 1.23E+04 |

### Example 2

An additional example of data measured and calculated by the system described herein is in Table 2 below. Newborn Calf Serum commercially obtained was utilized as the target fluid. The variable pitch device with variable pitch platform, as disclosed in U.S. Pat. No. 7,736,494, was employed as the gas-fluid contacting device. The following initial conditions were utilized; 600 ppmv ozone inlet concentration, 137 ml initial fluid volume, 1000 ml per minute gaseous flow rate, 189 ml per minute fluid flow rate counter current to the ozone/oxygen admixture flow. Incremental reductions in fluid volume are due to sampling of fluid through the fluid access port.

TABLE 2
NEWBORN CALF SERUM
MEASURED VARIABLES

| | | | | Average Inlet Ozone | Average Exit Ozone |
|---|---|---|---|---|---|
| Elapsed-time | Fluid Volume | Gas Flow Rate | Fluid Flow Rate | Concentration | Concentration |
| (5 minute intervals) | (milliliters) | (liters/minute) | (liters/minute) | (ppmv) | (ppmv) |
| 5 | 137 | 1.000 | 0.189 | 604.2 | 72.0 |
| 5 | 135 | 1.000 | 0.189 | 609.6 | 63.5 |
| 5 | 133 | 1.000 | 0.189 | 606.6 | 70.8 |
| 5 | 131 | 1.000 | 0.189 | 605.3 | 71.7 |

CALCULATED VARIABLES

| | Average | | | | |
|---|---|---|---|---|---|
| | Differential Ozone | | | Ozone Absorbed | Absorbed-dose |
| Elapsed-time | Concentration | Delivered-ozone | Residual-ozone | per Interval | of ozone |
| (minutes) | (ppmv) | (ug) | (ug) | (ug) | (ug) |
| 5 | 532.2 | 6.47E+03 | 7.70E+02 | 5.69E+03 | 5.69E+03 |
| 10 | 546.1 | 1.30E+04 | 1.45E+03 | 5.84E+03 | 1.15E+04 |
| 15 | 535.8 | 1.95E+04 | 2.21E+03 | 5.73E+03 | 1.73E+04 |
| 20 | 533.6 | 2.60E+04 | 2.98E+03 | 5.71E+03 | 2.30E+04 |

### Example 3

Another example of data measured and calculated by the system described herein is in Table 3 below. Newborn Calf Serum commercially obtained was utilized as the target fluid. The variable pitch device, as disclosed in U.S. Pat. No. 7,736,494, was employed as the gas-fluid contacting device. The following initial conditions were utilized; 900 ppmv ozone inlet concentration, 145 ml initial fluid volume, 1000 ml per minute gaseous flow rate, 189 ml per minute fluid flow rate counter current to the ozone/oxygen admixture flow. Incremental reductions in fluid volume are due to sampling of fluid through the fluid access port.

TABLE 1
NEWBORN CALF SERUM
MEASURED VARIABLES

| | | | | Average Inlet Ozone | Average Exit Ozone |
|---|---|---|---|---|---|
| Elapsed-time | Fluid Volume | Gas Flow Rate | Fluid Flow Rate | Concentration | Concentration |
| (5 minute intervals) | (milliliters) | (liters/minute) | (liters/minute) | (ppmv) | (ppmv) |
| 5 | 145 | 1.000 | 0.189 | 908.1 | 68.0 |
| 5 | 143 | 1.000 | 0.189 | 911.4 | 50.1 |
| 5 | 141 | 1.000 | 0.189 | 904.4 | 46.6 |
| 5 | 139 | 1.000 | 0.189 | 904.7 | 50.9 |

CALCULATED VARIABLES

| | Average | | | | |
|---|---|---|---|---|---|
| | Differential Ozone | | | Ozone Absorbed | Absorbed-dose |
| Elapsed-time | Concentration | Delivered-ozone | Residual-ozone | per Interval | of ozone |
| (minutes) | (ppmv) | (ug) | (ug) | (ug) | (ug) |
| 5 | 840.1 | 9.72E+03 | 7.28E+02 | 8.99E+03 | 8.99E+03 |
| 10 | 861.3 | 1.95E+04 | 1.26E+03 | 9.22E+03 | 1.82E+04 |
| 15 | 857.8 | 2.92E+04 | 1.76E+03 | 9.18E+03 | 2.74E+04 |
| 20 | 853.8 | 3.88E+04 | 2.31E+03 | 9.13E+03 | 3.65E+04 |

In one embodiment a method is provided to treat inflammatory disorders in a mammal comprising subjecting an amount of blood, blood fractionate, or other biological fluid ex vivo to an amount of ozone delivered by an ozone delivery system. The method may also provide for the maintenance of the biological integrity of the treated fluid. The method further comprises treatment conditions for fluid use in the treatment of inflammatory disorders at temperatures compatible with maintaining the biological integrity of biological fluids.

For blood or blood fractionates, the biological integrity of plasma may be measured by the functionality of its protein components either in whole plasma or after separation into plasma fractions. The biological integrity of red blood cell and platelet preparations may be determined by the methods and criteria known by those skilled in the art and are similar to those used in establishing the suitability of storage and handling protocols. In practical terms, the biological integrity of a biological fluid is a fluid that, subsequent to the method of treating for use in the treatment of inflammatory disorders described herein, has sufficiently maintained its functionality upon reinfusion into a mammalian patient.

Fluid-contacting surfaces including gas-fluid contacting devices constructed from ozone-inert material, may be treated with a human serum albumin (HSA) solution to prevent platelet adhesion, aggregation and other related platelet phenomena in the instances when a biological fluid to be treated contains platelets (i.e. whole blood, blood fractionates, or platelet concentrates). Generally, HSA solutions ranging between 1 and 10% may be employed. An HSA solution prepared in a biocompatible bacteriostatic buffer solution will be passaged throughout the gas-fluid contacting device. Subsequent to passage, the HSA solution will be drained from the device. The gas-fluid contacting device and all surfaces that are in contact with the biological fluid during the method described are consequently primed for use with platelet-containing biological fluids.

In alternative embodiments of the methods described herein, removal of blood directly from a subject and reinfusing it to the same patient occurs in a continuous loop configuration. The blood may circulate through the loop, which includes at least one gas-fluid contacting device, one or more times, wherein a measured amount of ozone is delivered to the blood, resulting in the absorption of a quantifiable absorbed dose of ozone, under conditions which maintain the biological integrity of the blood. The treated blood is constantly being reinfused directly back into the same patient, and contains a quantifiable absorbed-dose of ozone.

In another alternative embodiment of the methods described herein, the methods may comprise plasmapheresis wherein the patient's plasma is selectively removed while the balance of the blood cells is immediately returned to the patient. A measured amount of ozone is then delivered to the isolated plasma, resulting in the absorption of a quantifiable absorbed dose of ozone, under conditions which maintains the biological integrity of the plasma. The treated plasma is subsequently re-infused into the subject, and contains a quantifiable absorbed-dose of ozone.

In an alternative embodiment of the invention, the method comprises leukophoresis wherein the patient's white blood cells are selectively removed while the balance of the plasma is immediately returned to the patient. A measured amount of ozone is delivered to the isolated white blood cells, resulting in the absorption of a quantifiable absorbed dose of ozone, under conditions which induce apoptosis. The treated white blood cells are subsequently reinfused into the subject and contain a quantifiable absorbed-dose of ozone. In a further alternative embodiment, the method of leukophoresis to induce apoptosis in carried out in conditions to prevent excessive necrosis.

In those embodiments of the invention where the method of treatment involves a continuous loop methodology, the volume of blood treated can range between 10 ml and the total estimated circulating blood volume of a mammalian patient being treated multiple times. Generally, the blood volume treated will range between 10 ml and 10,000 ml, and preferably range between 10 ml and 6,000 ml.

The time required for an individual treatment through the use of a continuous loop format is based on a number of factors including the desired number of passes through the loop, volume of the fluid treated, the flow rate at which the fluid is circulating, the interface time required between the fluid and the amount of delivered-ozone, and the amount of the quantifiable absorbed-dose of ozone required. The time for the treatment can range from 1 minute to 720 minutes and preferably range from 1 minute to 180 minutes.

The number and frequency of treatments can vary considerably based upon the clinical situation of a particular patient. Generally the number of treatments can range between an individual treatment and 200 treatments, to be provided on a daily, alternate day or other schedule based on the clinical evaluation of the patient and desired clinical outcomes. Upon completion of a number of treatments and evaluation by a health care practitioner, another course of treatments may be indicated.

The methods of the present invention are directed to therapeutic treatment of inflammatory conditions and symptoms related thereto, comprising methods that employ ozone delivery devices that are constructed with all ozone-contacting surfaces being made or constructed of ozone-inert materials to assure accurate determination of the amount of ozone delivered to a fluid being treated, and to assure accurate determination of the amount of ozone absorbed by the fluid. The ozone delivery structures and related methods to treat blood and other biological fluids with ozone, and the use of those fluids for therapeutic treatments as disclosed herein, may be varied from those described to adapt them to specific applications. Therefore, reference to specific constructions and methods of use are by way of example and not by way of limitation.

What is claimed is:

1. A method for treating a mammalian subject suffering from, or believed to suffer from, inflammatory disease, comprising:
providing a biological fluid withdrawn from a mammalian subject;
processing said fluid in an ozone delivery system to deliver to said fluid a measured amount of ozone to effect absorption by the fluid of a quantifiable absorbed dose of ozone; and
reintroducing the treated fluid having a quantifiable absorbed dose of ozone to the mammalian subject to provide therapeutic treatment of inflammatory disease and conditions and symptoms thereof.

2. The method according to claim 1, wherein said processing of the fluid is carried out in an ozone delivery system all gas-contacting surfaces of which are constructed of ozone-inert materials.

3. The method according to claim 1, wherein said processing of the fluid is carried out in a discontinuous loop format.

4. The method according to claim 1, wherein said processing of the fluid is carried out in a continuous flow format.

5. The method according to claim 1, wherein said biological fluid is blood, a blood derivative or a blood fractionate.

6. The method according to claim 5, wherein said blood fractionate comprises separated cellular fractions or platelets.

7. The method according to claim 1, wherein said processing of said biological fluid is carried out in a manner to maintain the biological integrity of said fluid.

8. The method according to claim 1, wherein said therapeutic treatment further comprises eliciting a reduction in pro-inflammatory and/or an increase in anti-inflammatory cytokines released by immunomodulatory T cells.

9. The method according to claim 1, wherein said therapeutic treatment further comprises inducing sufficient leukocyte apoptosis, without excessive necrosis, to elicit clinical benefits.

10. The method according to claim 1, wherein the therapeutic treatment provides beneficial effects in anti-inflammatory response, neovascularization and vasodilation, vasorelaxatory effects and combinations of these effects.

11. The method according to claim 1, wherein the therapeutic treatment elicits increase in vasodilation, increased blood flow and combinations of these effects.

12. A method of producing a therapeutic substance for the treatment of inflammatory disease and related symptoms or conditions, comprising:
providing a biological fluid;
delivering to the biological fluid a measured amount of ozone to produce a therapeutic substance having a quantifiable absorbed-dose of ozone which, upon administration to a subject suffering, or believed to be suffering, from inflammatory disease, effectively treats the symptoms and conditions related to the inflammatory disease.

13. The method according to claim 12, wherein said biological fluid is blood, a blood derivative or blood fractionate.

14. A medicament for the treatment of inflammatory disease, and the symptoms or conditions related thereto, comprising a biological fluid containing a quantifiable absorbed-dose of ozone to provide efficacious therapeutic effect to a subject suffering, or believed to be suffering, from inflammatory disease and the symptoms or conditions related thereto upon administration of the medicament to the subject.

15. The medicament according to claim 14, wherein said biological fluid is blood, a blood derivative or blood fractionate.

16. The medicament according to claim 15, wherein said blood fractionate is comprised of platelets.

17. The medicament according to claim 15, wherein said blood derivative is plasma.

18. The medicament according to claim 15, wherein the conditions and symptoms of inflammatory disease that effectively treated by the medicament include rheumatoid arthritis, multiple sclerosis, systemic lupus erythromatosis (SLE), scleroderma, diabetes, inflammatory bowel disease, psoriasis, pemphigus, atherosclerosis, chronic heart failure, graft versus host reactions including tissue transplant rejection, Alzheimer's disease, ischemic brain stroke, senile dementia, depression, Down's syndrome, Huntington's disease, peripheral neuropathies, spinal cord diseases, neuropathic joint diseases, chronic inflammatory demyelinating disease (CIPD) and neuropathies, including mononeuropathy, polyneuropathy, symmetrical distal sensory neuropathy, cystic fibrosis, neuromuscular junction disorders, myasthenias, Parkinson's disease, traumatic brain injury, spinal cord injury and soft tissue injuries.
